(19) United States
(12) Patent Application Publication
(10) Pub. No.: US 2003/0126627 A1
(43) Pub. Date: Jul. 3, 2003

(54) Transgenic mouse model of inclusion body myositis

(76) Inventor: Frank M. LaFerla, Irvine, CA (US)

Correspondence Address: CHRISTIE, PARKER & HALE, LLP, P.O. BOX 7068, PASADENA, CA, 91109-7068, US

(21) Appl. No.: 10/288,138

(22) Filed: Nov. 4, 2002

Related U.S. Application Data
(60) Provisional application No. 60/338,832, filed on Nov. 5, 2001.

(51) Int. Cl. A01K 67/027

(52) U.S. Cl. 800/9; 800/18

(57) ABSTRACT

Inclusion body myositis (IBM), the most common age-related muscle disease in the elderly population, is an incurable disorder leading to severe disability. Sporadic IBM has an unknown etiology, although affected muscles fibers are characterized by many of the pathobiochemical alterations traditionally associated with neurodegenerative brain disorders such as Alzheimer's disease (AD). Accumulation of the amyloid-β peptide (Aβ), which is derived from proteolysis of the larger amyloid-β precursor protein (βAPP), appears to be an early pathological event in AD and also in IBM, where in the latter, it occurs predominantly intracellularly within affected myofibers. To elucidate the possible role of βAPP mismetabolism in the pathogenesis of IBM, transgenic mice were derived in which βAPP overexpression was selectively targeted to skeletal muscle using the muscle creatine kinase promoter. Skeletal muscle from transgenic mice older than 10 months was shown to contain intracellular immunoreactivity to βAPP and its proteolytic derivatives, which was quantifiable by ELISA. In this transgenic model, selective overexpression of βAPP leads to the development of a subset of other histopathological and clinical features characteristic of IBM, including centric nuclei, inflammation, and deficiencies in motor performance.

### CROSS-REFERENCE TO RELATED APPLICATION(S)

This application claims the benefit of U.S. Provisional Application Ser. No. 60/338,832, entitled “Transgenic Mouse Model of Inclusion Body Myositis,” filed Nov. 5, 2001, the contents of which are hereby incorporated by reference in its entirety.

### STATEMENT REGARDING FEDERALLY SPONSORED RESEARCH OR DEVELOPMENT

This invention was made with Government support under Grant No. NIH R29-AG1509, awarded by the National Institutes of Health. The Government may have certain rights in this invention.

### BACKGROUND OF THE INVENTION

Brain and skeletal muscle are the only two known tissues in humans marked by the pathological accumulation of the highly amyloidogenic amyloid-β (Aβ) peptide. In brain, Aβ deposition is associated with several genetically-related neurodegenerative disorders including Alzheimer's disease (AD), Down syndrome, and hereditary cerebral hemorrhage with amyloidosis-Dutch type. Selkoe, D. J. (2001) Physiol Rev 81: 741-66. Based on genetic evidence Aβ accumulation appears to be an early pathogenic event, although it remains to be determined whether Aβ directly leads to cell degeneration or if this is carried out by other downstream factors induced by it. In muscle, Aβ accumulation is associated with inclusion body myositis (IBM), the most common muscle disorder to afflict the elderly. IBM is, therefore, the first human disorder marked by the pathological accumulation of this amyloidogenic peptide outside the CNS. Notably, Aβ and/or other Aβ-containing fragments produced by proteolysis of the amyloid-β precursor protein (βAPP) are not implicated in other myopathies, suggesting that βAPP mismetabolism is an integral component of the molecular pathogenesis of IBM.

Like AD, IBM is an age-related degenerative disorder with a slowly progressive clinical course for which no effective treatment is available. Clinically characterized by muscle weakness and atrophy involving both proximal and distal muscle groups of the limbs (Dalakas, M. C. (1992) Clin. Neuropharmacol. 15: 327-51; Oldfors, A. & Lindberg, C. (1999) Curr. Opin. Neurol. 12: 527-33; Askanas, V. & Engel, W. K. (1993) Curr. Opin. Rheumatol. 5: 732-41), IBM was first recognized as its own disorder in the early 1970s (Yunis, E. J. & Samaha, F. J. (1971) Lab. Invest. 25:240-8). Prior to that time, IBM was often diagnosed as polymyositis. Amato, A. A. & Barohn, R. J. (1997) Neurol. Clin. 15:615-48. That Aβ-containing fragments are involved in the pathogenesis of IBM is somewhat surprising because no obvious genetic link exists to either the βAPP gene or to other AD-related genes such as apolipoprotein E. Askanas, V., et al. (1996) Ann. Neurol. 40: 264-5; Harrington, C. R., Anderson, J. R. & Chan, K. K. (1995) Neurosci. Lett. 183: 35-8. Nevertheless, IBM and AD share many pathobiochemical features including the occurrence of twisted intracellular tubulofilaments consisting of hyperphosphorylated tau (Askanas, V., et al. (1994) Am. J. Pathol. 144:177-87) and the aberrant accumulation of other “dementia”-related proteins, including apoE, presenilin, prion protein, and alpha-synuclein (Askanas, V., et al. (1994) Lancet 343:364-5; Askanas, V., et al. (1993) Neuroreport 5:25-8; Askanas, V., et al. (1998) Am. J. Pathol. 152: 889-95; Askanas, V., et al. (2000) J. Neuropathol. Exp. Neurol. 59: 592-8). These data suggest that following an initial insult, a coordinated molecular cascade occurs, triggering the accumulation of these “dementia”-related proteins both in muscle and in brain. Along these lines, AD patients also contain slightly elevated levels of amyloidogenic Aβ₁₋₄₂ peptides in their muscle but this seems to be without pathological consequence, perhaps due to their low levels. Kuo, Y. M., et al. (2000) Am. J. Pathol. 156:797-805. Curiously, it has recently been reported that myoglobin can also form amyloid fibrils, but whether this plays a role in muscle disease is not yet established. Fandrich, M., Fletcher, M. A. & Dobson, C. M. (2001) Nature 410: 165-6.

One interesting distinction between IBM and AD involves the location of the Aβ deposits. Whereas the AD brain is predominantly characterized by accumulation of amyloid deposits in extracellular plaques, Aβ accumulates intracellularly in IBM. Askanas, V., Engel, W. K. & Alvarez, R. B. (1992) Am. J. Pathol. 141: 31-6. Although there are reports that Aβ accumulates intraneuronally (LaFerla, F. M., et al. (1997) J. Clin. Invest. 100: 310-20; Gouras, G. K., et al. (2000) Am. J. Pathol. 156: 15-20), it has not yet been established whether this intracellular form of Aβ is relevant in the neurodegenerative cascade. Despite this cytogeographical difference, Aβ-containing fragments appear to play a critical pathogenic role in IBM. Whereas increased expression of βAPP, aberrant proteolysis, and/or diminished clearance of Aβ-containing fragments in muscle could each contribute to amyloid accumulation in IBM muscle fibers, there is clear evidence of excessive βAPP transcripts in IBM. Sarkozi, E., et al. (1993) Neuroreport 4:815-8. Moreover, the subcellular distribution of βAPP appears to be altered in IBM myofibers, away from the postsynaptic domain of the neuromuscular junction to a subsarcolemmal location in IBM fibers. Id. Further underscoring the potential pathological consequences of βAPP overexpression in muscle, it has been shown that transfection of normal cultured muscle cells with βAPP mRNA leads to IBM-like changes including Congo red positive amyloid and cytoplasmic tubulofilaments (Askanas, V., et al. (1996) Proc. Natl. Acad. Sci. 93: 1314-9; Askanas, V., et al. (1997) Neuroreport 8:2155-8), further implicating βAPP overexpression as an early pathological event.

Transgenic mouse models incorporating human βAPP have been developed. These models express a truncated version of the protein, rather than a full-length amyloid-β precursor protein. However, the physiological significance of overexpressing truncated protein is unclear, and it bypasses the inherent processing effects that the full-length protein would undergo, as well as the normal cellular trafficking. Also, expression of prior art transgenes is not muscle-specific, as a gene promoter was used that leads to ubiquitous expression of the transgene in virtually every tissue in the body. Because IBM is a muscle disorder and doesn't appear to affect other tissue types, a mouse model with muscle-specific expression, rather than ubiquitous expression, would be preferred.

What is needed, therefore, is a transgenic mouse model in which the full length amyloid-β precursor protein is expressed, resulting in a more physiologically relevant model. What is further needed is a transgenic mouse model in which transgene expression is muscle specific. Such a transgenic mouse model would be a valuable tool for use in trials of potential therapeutic agents aimed at treating inclusion body myositis. Such transgenic mice would also be valuable for basic research investigations aimed at understanding the behavioral, physiological, molecular/cell biological, and pharmacological processes underlying this muscle disease in an animal model.

### SUMMARY OF THE INVENTION

One aspect of the invention is directed to a transgenic mouse whose genome comprises a transgene encoding human β-amyloid precursor protein (“βAPP”), wherein the transgene is operably linked to a muscle-specific promoter, and wherein expression of the transgene results in increased formation of Aβ-containing βAPP proteolytic fragments.

Another aspect of the invention is directed to a transgenic mouse whose genome comprises a transgene encoding full-length β-amyloid precursor protein, wherein the transgene is operably linked to a muscle-specific promoter, and wherein expression of the transgene results in a neutrophil-mediated inflammatory response clustered around βAPP immunoreactive regions.

Another aspect of the invention is directed to a transgenic mouse whose genome comprises a transgene encoding full-length β-amyloid precursor protein, wherein the transgene is operably linked to a muscle-specific promoter, and wherein expression of the transgene results in deficits in motor performance.

Another aspect of the invention is directed to a method of screening biologically active agents that facilitate reduction of Aβ-containing βAPP proteolytic fragments in vivo, the method comprising administering a candidate agent to a transgenic mouse whose genome comprises a transgene encoding βAPP, wherein the transgene is operably linked to a muscle-specific promoter, and wherein expression of the transgene results in increased formation of Aβ-containing βAPP proteolytic fragments, and determining the effect of said agent upon the amount of Aβ-containing βAPP proteolytic fragments.

Another aspect of the invention is directed to a method of screening biologically active agents that facilitate reduction of a neutrophil-mediated inflammatory response clustered around βAPP immunoreactive regions in vivo, the method comprising administering a candidate agent to a transgenic mouse whose genome comprises a transgene encoding full-length β-amyloid precursor protein, wherein the transgene is operably linked to a muscle-specific promoter, and wherein expression of the transgene results in a neutrophil-mediated inflammatory response clustered around βAPP immunoreactive regions, and determining the effect of said agent upon the amount of the neutrophil-mediated inflammatory response.

Another aspect of the invention is directed to a method of screening biologically active agents that facilitate improvement in motor performance, the method comprising administering a candidate agent to a transgenic mouse whose genome comprises a transgene encoding full-length β-amyloid precursor protein, wherein the transgene is operably linked to a muscle-specific promoter, and wherein expression of the transgene results in deficits in motor performance, and determining the effect of said agent upon motor performance.

### BRIEF DESCRIPTION OF THE DRAWINGS

FIG. 1. Transgene map and expression analysis. (A) The MCK promoter was used to target expression of human βAPP cDNA to skeletal muscle. The approximately 3.75-kb transgenic construct was isolated by BssHII restriction digestion; also shown is the BamHI site used in the Southern blot analysis. (B) Northern blot analysis of skeletal muscle RNA from transgenic mice reveals overexpression of the βAPP transgene in both the A2 and A6 lines. The arrow points to the band corresponding to transgene mRNA. Note that no endogenous βAPP mRNA is detected since the SV40 probe was used. Stripping and reprobing this same blot for β-actin mRNA revealed nearly equivalent RNA loading, indicating that the variation in transgene band intensities accurately reflects differences in transgene expression levels between the two lines. Densitometric analysis reveals approximately l2-times more βAPP transgene mRNA in the A6 line compared to the A2 line. (C) Northern blot analysis of total RNA prepared from several tissues from an A6 mouse (10-weeks of age) shows that expression was selective for muscle with no expression detected in non-target tissues including brain, intestine, kidney, liver, lung or stomach. Only muscle expressed the transgene with levels approximately 8-times higher in skeletal muscle compared to cardiac muscle.

FIG. 2. Immunoblot analysis of human βAPP in skeletal muscle. Protein extracts from skeletal muscle were prepared from age-matched transgenic mice from the A2 and A6 lines along with a nontransgenic littermate. Both transgenic lines clearly show accumulation of the human βAPP protein while, as expected, no signal was detected in the nontransgenic littermate. Densitometric analysis reveals the A6 line expresses approximately 9-times the amount of human βAPP, compared to the A2 line. Amount of protein loaded was nearly equivalent as measured by immunoblotting for β-actin.

FIG. 3. IBM histopathological changes in MCK-βAPP skeletal muscle. (A) HE staining of nontransgenic mouse skeletal muscle reveals fibers uniform in size containing peripherally located nuclei. (B) Myofibers in aged MCK-βAPP mice harbor multiple centric nuclei, which is indicative of underlying pathology. (C) HE staining of MCK-βAPP skeletal muscle also reveals variable fiber size and focal regions containing strongly basophilic mononuclear cells that strongly correlates with (E) the degree of intracellular Aβ immunoreactivity. The pattern of intracellular Aβ accumulation occurred as (E and H) diffuse amorphous deposits as well as (G) circular granular patterns, none of which were present in (F) immunostained control tissue. (D) Neutrophils were identified as the predominant inflammatory cell type. Skeletal muscle sections of transgenic mice are shown: B, A2 line (15 months); C, E A2 line (16 months); D, A2 line (14 months); G, A6 line (12 months); H, A6 line (14 months). Nontransgenic sections: A, (15 months); F, (16 months). Original magnifications: A, B, 100×; C through H, 400×.

FIG. 4. MCK-βAPP mice display an age-related decrease in motor performance on the accelerating rotarod. As MCK-βAPP mice age, their ability to remain on the rotarod diminishes compared to age-matched nontransgenic littermates. These differences were significant for aged transgenic mice in the A6 line compared to controls at 15 to 20 and 20 to 25 months of age (p<0.05, Student's t-test). Transgenic mice from the A2 line display a trend towards an age-related decline in motor performance as well, although these data were not statistically significant. Error bars represent the standard error of the mean. Mice between 26 and 34 grams were used in the analyses. Number of mice evaluated were as follows: A2 and [A6] transgenic mice 0-5 months n=6 [6]; 5-10 months n=6 [6]; 10-15 months n=4 [5]; 15-20 months n=4 [6]; 20-25 months n=9 [8]. Age-matched nontransgenic littermates were used as controls: 0-5 months n=6; 5-10 months n=6; 10-15 months n=6; 15-20 months n=6; 20-25 months n=8.

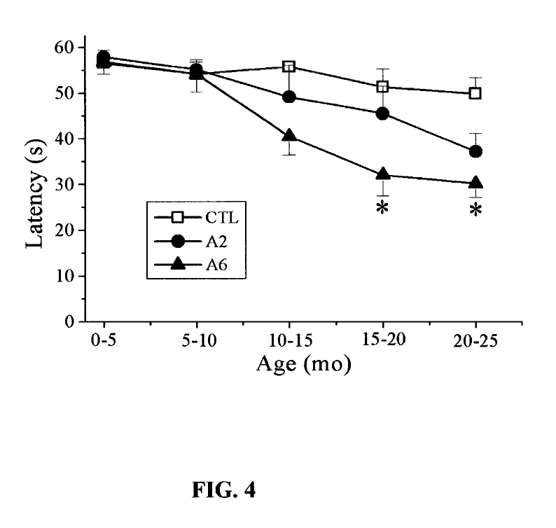

FIG. 5. Schematic diagram of isolation protocol for analyzing protein levels in individual myofibers. Sol, Pl and the WMG were dissected from MCK-βAPP transgenic mice and individual myofibers were isolated by microdissection. One half of each fiber was used to determine steady-state levels of transgene protein by immunoblotting with a human βAPP specific antibody. These blots were then probed for β-actin, which was used to normalize transgene protein levels. The other halves of the fibers were used to determine MHC isoform composition, thereby providing a method to identify the fiber type (i.e. Type I or IIB).

FIG. 6. Relative levels of MHC isoforms in Sol, Pl and WMG muscles. Total protein extracted from Sol (S), Pl (P) and WMG (W) muscles of MCK-βAPP transgenic mice was electrophoresed to separate out MHC isoforms based on size. The left panel (A) shows a representative gel revealing the four distinct MHC isoforms. The right panel (B) illustrates the relative proportion of each MHC isoform found in a given muscle. Note the heterogeneity within the Sol and relative high percentage of Type IIB in the Pl and WMG. Each bar represents an n=5 (five different mice), and error bars are standard error of the mean.

FIG. 7. βAPP expression in different muscle groups. (A) Immunoblotting for human βAPP in Sol (S), Pl (P) and WMG (W) in MCK-βAPP mice revealed higher levels of βAPP in the Pl and WMG compared to the Sol. (B) Densitometric analysis of band intensities after normalizing to β-actin revealed approximately 8-times the level of transgene protein in the Pl and WMG compared to Sol. Error bars represent standard error of the mean between 5 separate transgenic mice; Non-Tg=nontransgenic littermate control muscle.

FIG. 8. Single-fiber analysis for human βAPP. A representative immunoblot shown in the top panel reveals four out of five Pl single myofibers contain full-length human βAPP (A). β-actin immunoreactivity confirms successful isolation of a fiber in all five samples and was also used to normalize protein levels (A). MHC isoform analyses demonstrated each of these were fast Type IIB fibers (B).

FIG. 9. Histograms illustrating relative steady-state levels of human βAPP in single Type I and Type IIB fibers. The ratio of human βAPP to β-actin was analyzed as a function of fiber type. As shown in the top panel, Type I fibers either contained low levels human βAPP or none at all. Relatively more single Type IIB fibers contained higher levels of βAPP as shown in the bottom panel.

FIG. 10. Immunohistochemical localization of full-length transgene human βAPP in transgenic mouse muscles. Cryosections from a 9-mo old A6 MCK-βAPP mouse were immunostained with P2-l antibody. Because this antibody is human-specific, the Sol and Pl muscles of an age-matched nontransgenic control (cf, A, C) do not reveal any immunoreactivity. The transgenic Pl muscle demonstrates strong immunoreactivity with numerous fibers staining with varying intensities (D). Although protein extracts from the whole Sol muscle of a transgenic-revealed low levels of transgene protein by Western blotting, it was not detected via immunohistochemistry (B).

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Definitions

The term “transgene” refers to the genetic material which has been or is about to be artificially inserted into the genome of an animal, particularly a mammal and more particularly a mammalian cell of a living animal.

“Transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

“Operably linked” means that a DNA sequence and a regulatory sequence(s) are connected in such a way as to permit gene expression when the appropriate transcriptional activator proteins are bound to the regulatory sequence(s). For example, a nucleic acid sequence encoding amyloid-β precursor protein (βAPP) may be operably linked to a skeletal muscle-specific promoter to facilitate production of βAPP polypeptide in muscle cells.

“Skeletal muscle-specific promoter” refers to a regulatory sequence(s) operably linked to a βAPP-encoding nucleic acid in such a way as to permit gene expression of the βAPP-encoding nucleic acid in skeletal muscle cells with little or no expression of the βAPP-encoding nucleic acid in non-skeletal muscle cells. One such skeletal muscle-specific promoter is the muscle creatine kinase promoter; other examples are known to those of skill in the art.

“Inclusion body myositis” (abbreviated herein as “IBM”) refers to a condition associated with βAPP accumulation in skeletal muscle fibers, as well as muscle degeneration, inflammation, and age-related decline in motor performance. “IBM” as used herein is meant to encompass both IBM as well as IBM-type pathologies.

Overview of the Invention

The invention provides non-human transgenic animal models useful for evaluating various therapies for the treatment of inclusion body myositis (IBM) patients and for studying the molecular pathogenesis of IBM. IBM is the most common age-related muscle disorder in humans over 50 years of age. Surprisingly, IBM shares several pathological features with Alzheimer's disease (AD), a common neurodegenerative disorder that leads to progressive memory loss. Although IBM patients do not suffer from memory loss, there is a relationship between this muscle disease and Alzheimer's disease because the pathological proteins that are characteristic of Alzheimer's disease pathology accumulate in the skeletal muscle fibers in IBM. It appears that the most pathologically relevant protein that accumulates in their muscle fibers is the βAPP that leads to the hallmark plaque structures that are characteristic of AD neuropathology.

The animals of the present invention are genetically altered so as to overexpress human βAPP in a tissue-specific manner. Specifically, expression of full-length βAPP is targeted to skeletal muscle fibers using skeletal muscle-specific promoters, such as the muscle creatine kinase promoter. The transgenic animals may be either homozygous or heterozygous for the genetic alteration.

The transgenic animals of the present invention exhibit several hallmark features of IBM, including prominent βAPP accumulation in skeletal muscle fibers, muscle degeneration, inflammation, and age-related decline in motor performance. Given that IBM is the most common muscle disorder in aged humans and that there are no effective treatments, this transgenic animal model represents a valuable research tool for testing candidate agents for treatment of individuals diagnosed with IBM, either prophylactically or after disease onset.

Transgenic Animals

Transgenic animals comprise exogenous DNA incorporated into the animal's cells to effect a permanent or transient genetic change, preferably a permanent genetic change. Permanent genetic change is generally achieved by introduction of the DNA into the genome of the cell. Vectors for stable integration include plasmids, retroviruses and other animal viruses, YACs, and the like. Generally, transgenic animals are mammals, most typically mice.

The exogenous nucleic acid sequence may be present as an extrachromosomal element or stably integrated in all or a portion of the animal's cells, especially in germ cells. Unless otherwise indicated, a transgenic animal comprises stable changes to the germline sequence. During the initial construction of the animal, chimeric animals (chimeras) are generated, in which only a subset of cells have the altered genome. Chimeras may then be bred to generate offspring heterozygous for the transgene. Male and female heterozygotes are may then be bred to generate homozygous transgenic animals.

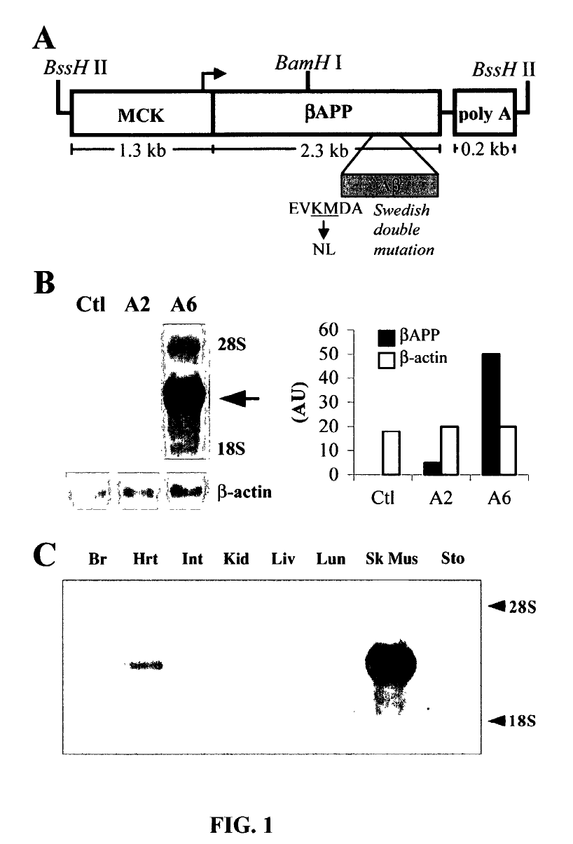

Typically, transgenic animals are generated using transgenes from a different species or transgenes with an altered nucleic acid sequence. For example, a human gene, such as the nucleic acid encoding βAPP, may be introduced as a transgene into the genome of a mouse. The introduced gene may be a wild-type gene, naturally occurring polymorphism, or a genetically manipulated sequence, for example having deletions, substitutions or insertions in the coding or non-coding regions. For example, the introduced human βAPP gene may be wild type or may include a mutation such as the “Swedish mutation” as shown in FIG. 1. Where the introduced gene is a coding sequence, it is usually operably linked to a promoter, which may be constitutive or inducible, and other regulatory sequences required for expression in the host animal.

In general, the transgenic animals of the invention comprise transgenes that express βAPP, preferably human βAPP. Preferably, the introduced sequences provide for high expression of βAPP. Specific βAPP-encoding constructs of interest are described below. Of particular interest and importance is the expression of a full-length form of βAPP, preferably skeletal muscle-specific expression of full-length βAPP.

The transgenic animals of the invention can comprise other genetic alterations in addition to the presence of the βAPP-encoding sequences. For example, the host's genome may be altered to affect the function of endogenous genes (e.g., endogenous APP-encoding genes), contain marker genes, or other genetic alterations consistent with the goals of the present invention.

Nucleic Acid Compositions

Constructs for use in the present invention include any construct suitable for use in the generation of transgenic animals having the desired levels of expression of a desired βAPP-encoding sequence. Methods for isolating and cloning a desired sequence, as well as suitable constructs for expression of a selected sequence in a host animal, are well known in the art. In addition to the βAPP-encoding sequences, the construct may contain other sequences, such as a detectable marker.

The βAPP-encoding construct can contain a wild-type sequence encoding βAPP or mutant forms of βAPP, including nucleotide insertions, deletions, splice variants, and base substitutions, especially those associated with IBM and/or IBM-type pathologies in humans. The βAPP-encoding construct may include the open reading frame encoding specific polypeptides, introns, and adjacent 5′ and 3′ non-coding nucleotide sequences involved in the regulation of expression. The DNA sequences encoding βAPP may be cDNA or genomic DNA or a fragment thereof. The genes may be introduced into an appropriate vector for extrachromosomal maintenance or for integration into the host.

The nucleic acid compositions used in the subject invention may encode all or a part of βAPP as appropriate. Fragments may be obtained of the DNA sequence by chemically synthesizing oligonucleotides in accordance with conventional methods, by restriction enzyme digestion, by PCR amplification, and by other techniques known in the art.

Several isoforms and homologs of βAPP are known. Additional homologs of cloned βAPP are identified by various methods known in the art. For example, nucleic acids having sequence similarity are detected by hybridization under low stringency conditions. Labeled nucleotide fragments can be used to identify homologous βAPP sequences as, for example, from other species.

The βAPP gene and exemplary derivatives thereof suitable for use in the production of the transgenic animals of the invention are described below.

The APP Gene and its Derivatives Suitable for Use in the Present Invention

The APP gene has been described in U.S. Pat. No. 6,455,757, the entire contents of which are hereby incorporated by reference.

Transgenic animals of the present invention comprise a heterologous sequence encoding a desired APP gene, preferably a human βAPP gene. Preferably, the host animal produces high levels of human βAPP or its proteolytic fragments, such as human Aβ₄₂, in skeletal muscle. Preferably, the βAPP gene encodes a genomic βAPP sequence or a sequence encoding a spliced βAPP gene (e.g., a cDNA), more preferably a full-length human βAPP cDNA sequence. Alternatively, the βAPP gene can be an mutant, particularly an βAPP mutant associated with IBM and/or an IBM-type pathology. Mutants of particular interest include human βAPP cDNA harboring the Swedish double mutation.

The host animals can be homozygous or heterozygous for the βAPP-encoding sequence, preferably homozygous. The βAPP gene can also be operably linked to a promoter to provide for a desired level of expression in the host animal and/or for tissue-specific expression. Preferably, βAPP gene expression is driven by a skeletal muscle-specific promoter, preferably a muscle creatine kinase promoter. Expression of βAPP can be either constitutive or inducible.

βAPP genes suitable for use in the present invention have been isolated and sequenced. The sequence for human β-amyloid precursor protein is found at GenBank Accession No. XM047793. See also, Table 2 of U.S. Pat. No. 6,455,757, providing a list of human APP sequences with Genbank accession numbers relating to the listed APP sequences.

Methods of Making Transgenic Animals

Transgenic animals can be produced by any suitable method known in the art, such as manipulation of embryos, embryonic stem cells, etc. Transgenic animals may be made through homologous recombination, where the endogenous locus is altered. Alternatively, a nucleic acid construct is randomly integrated into the genome. Vectors for stable integration include plasmids, retroviruses and other animal viruses, YACs, and the like.

Specific methods of preparing the transgenic animals of the invention as described herein. However, numerous methods for preparing transgenic animals are now known and others will likely be developed. See, e.g., U.S. Pats. Nos. 6,252,131, 6,455,757, 6,028,245, and 5,766,879, all incorporated herein by reference. Any method that produces a transgenic animal expressing βAPP in muscle cells is suitable for use in the practice of the present invention. The microinjection technique described is particularly useful for incorporating transgenes into the genome without the accompanying removal of other genes.

Drug Screening Assays

The transgenic animals described herein may be used to identify compounds useful in the treatment of IBM and/or IBM-related pathologies. For example, transgenic animals of the present invention may be treated with various candidate compounds and the resulting effect, if any, on βAPP accumulation in skeletal muscle fibers, muscle degeneration, inflammation, and/or age-related decline in motor performance evaluated. Preferably, the compounds screened are suitable for use in humans.

Drug screening assays in general suitable for use with transgenic animals are known. See, for example, U.S. Pats. Nos. 6,028,245 and 6,455,757. Immunoblot analyses, expression studies, measurement of Aβ proteolytic fragments by ELISA, immunocytochemical and histological analysis of skeletal muscle sections and behavioral analyses suitable for use with the transgenic animal of the present invention are described herein. However, it will be understood by one of skill in the art that many other assays may also be used. The subject animals may be used by themselves, or in combination with control animals. Control animals may have, for example, a wild-type βAPP transgene that is not associated with IBM, or may be transgenic for a control construct that does not contain an βAPP-encoding sequence. The screen using the transgenic animals of the invention can employ any phenomena associated with IBM or IBM-related pathologies that can be readily assessed in an animal model.

Therapeutic Agents

Once compounds have been identified in drug screening assays as eliminating or ameliorating the effects of IBM and/or IBM-related pathologies, these compounds can be used as therapeutic agents, provided they are biocompatible with the animals, preferably humans, to whom they are administered.

The therapeutic agents of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. Administration of the compounds can be administered in a variety of ways known in the art, as, for example, by oral, buccal, rectal, parenteral, intraperitoneal, intradermal, transdermal, intratracheal, etc., administration.

Depending upon the particular route of administration, a variety of pharmaceutically acceptable carriers, well known in the art can be used. These carriers include, but are not limited to, sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, 16th Edition, Mack, (1980)).

The concentration of therapeutically active compound in the formulation may vary from about 0.1-100 wt. %.

Those of skill will readily appreciate that dose levels can vary as a function of the specific therapeutic agents, the severity of the symptoms and the susceptibility of the subject to side effects. Preferred dosages for a given therapeutic agent are readily determinable by those of skill in the art by a variety of means. A preferred means is to measure the physiological potency of a given therapeutic agent.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Inclusion Body Myositis-Like Phenotype Induced by Transgenic Overexpression of βAPP in Skeletal Muscle Materials and Methods

Generation of Transgenic Mice. Human βAPP cDNA harboring the Swedish double mutation was subcloned into the pBlueScript KSII cloning vector (Stratagene) downstream of the 1.3-kb 5′-flanking sequence of the muscle creatine kinase gene (MCK) and upstream of the SV40 polyadenylation signal (FIG. 1A). The 3.8-kb transgene was isolated from the cloning vector by digestion with BssHII and purified by sucrose gradient fractionation. After overnight dialysis in injection buffer (10 mM Tris, pH 7.5, 0.25 mM EDTA), the construct was microinjected into the pronuclei of single-cell embryos from C57BL6/SJL mice (Jackson Labs) at the Transgenic Mouse Facility at the University of California, Irvine. Transgenic mice were identified by Southern blot analysis of tail DNA isolated from 10-day-old pups using established procedures (LaFerla, F. M., et al. (1995) Nature Genet. 9: 21-30; LaFerla, F. M., et al. (2000) J. Mol. Neurosci. 15:45-59). DNA was restricted with BamHI, resolved by agarose gel electrophoresis, transferred and immobilized to nitrocellulose. The blot was hybridized with a random-primed ³²P-labeled probe that encompassed the entire transgene cassette. Transgenic lines were maintained as hemizygous strains so that nontransgenic control mice were produced in each litter.

Expression Analysis of Transgenic Mice. Hind limb skeletal muscles from 10-week old transgenic mice were mechanically homogenized and total RNA was isolated via the guanidinium isothiocyanate-acid phenol method (Chomczynski, P. & Sacchi, N. (1987) Anal. Biochem. 162: 156-9). RNA (10 μg) was loaded onto a denaturing agarose/formaldehyde gel and transferred to nitrocellulose. To label transgene mRNA only, Northern blots were hybridized with a random-primed ³²P-labeled 0.24-kb SV40 polyA DNA fragment. Blots were stripped and re-probed for β-actin mRNA to control for RNA loading. For quantification of band intensities, Phosphoimaging screens (BioRad) were scanned on the Storm system and densitometric analysis was carried out with Imagequant software.

Antibodies. Antibody sources were as follows: anti-Aβ (4G8 and 6E10; Senetec), (anti-Aβₓ₋₄₀ and anti-Aβₓ₋₄₂; BioSource International); anti-actin (anti-actin; Sigma); anti-CD3ε (anti-T-cell; Pharmingen); anti-CDl1b (anti-macrophage; Sigma); anti-neutrophil (Serotec).

Immunoblot Analysis. Skeletal muscle from the hind limb of transgenic mice was mechanically homogenized for 20 seconds in buffer containing 2.5% SDS supplemented with a Complete Mini Protease Inhibitor Tablet (Roche). To remove insoluble proteins, homogenized samples were spun at 100,000 g for 60 min at 80C. For βAPP and β-actin immunoblots, soluble protein extracts were resolved by SDS/PAGE (7.5% Tris-Acetate from Invitrogen) under reducing conditions, transferred to nitrocellulose and probed with monoclonal antibody 6E10 or anti-β-actin at dilutions of 1:500 and 1:1000, respectively. To solubilize any fibrillar Aβ, SDS-insoluble pellets were extracted in 70% formic acid and centrifuged at 100,000 g for 60 min at 8° C.

Immunocytochemical and Histological Analysis of Skeletal Muscle Sections. Formalin-fixed, paraffin-embedded muscle was sectioned at 10 μm and mounted on silane-coated slides and processed as previously described (LaFerla, F. M., et al. (1995) Nature Genet. 9: 21-30). For cryosections, skeletal muscle tissue was frozen in liquid nitrogen-cooled isopentane, sectioned at 10 μm and stored at −20° C. Primary antibodies were applied at dilutions of 1:1000 for 4G8 and 6E10; 1:500 for anti-Aβ₁₋₄₀ and anti-Aβ₁₋₄₂; 1:100 for anti-neutrophil, anti-CD3ε and anti-CD1lb. Sections were developed with diaminobenzidine (DAB) substrate using the avidin-biotin horseradish peroxidase system (Vector Labs).

Measurement of Aβ by ELISA. Aβ₁₋₄₀ and Aβ₁₋₄₂ were measured as previously described using the BAN50/BA27 and BAN50/BC05 ELISA system (Murphy, M. P., et al. (2000) J. Biol. Chem. 275: 26277-84; Murphy, M. P., et al. (1999) J. Biol. Chem. 274: 11914-23; Suzuki, N., et al. (1994) Science 264: 1336-40; Kuo, Y. M., et al. (1996) J. Biol. Chem. 271: 4077-81). SDS and formic acid fractions isolated from skeletal muscle as described above were used in the Aβ ELISAs. SDS fractions were diluted 1:40 in buffer (0.1 M NaH₂PO₄; 0.1 M Na₂HPO₄; 0.05% % NaN₃; 2 MM EDTA; 0.4 M NaCl; 1% BSA and 0.05% CHAPS) (Suzuki, N., et al. (1994) Science 264: 1336-40) and formic acid fractions were diluted 1:20 in 1M Tris base prior to loading onto ELISA plates. MaxiSorp immunoplates (Nunc) were coated with BAN50 at a concentration of 5 μg/ml in 0.1 M NaCO₃ buffer, pH 9.6, and blocked with 1% Block Ace (Snow Brand Milk Products; Sapporo, Japan). Synthetic Aβ standards, internal controls and samples were run at least in duplicate. After overnight incubation at 4° C., wells were probed with either HRP-conjugated BA27 (for Aβ₁₋₄₀) or BC05 (for Aβ₁₋₄₂) for 2-3 hours at 37° C. 3,3′,5,5′-tetramethylbenzidine was used as the chromagen, and the reaction was stopped by 6% 0-phosphoric acid, and read at 450 nm on a Molecular Dynamics plate reader. Numbers of mice used were as follows: nontransgenics, total n=11; A2, n=13; A6, n=13.

Immunodepletion of βAPP and βAPP C-terminal fragments, which includes full-length βAPP, C83 and C99, was performed as follows. SDS-extracted skeletal muscle tissue was diluted 1:20 in RIPA buffer and incubated overnight at 4° C. in the presence of 5 microliters of CT-20 antiserum (directed against the last 20 amino acids of βAPP (Murphy, M. P., et al. (2000) J. Biol. Chem. 275: 26277-84; Pinnix, I. et al. (2001) J. Biol. Chem. 276: 481-87) and 50 microliters of protein A agarose (Gibco BRL). The cleared extract was collected, loaded onto ELISA plates and read against synthetic Aβ standards prepared in the same buffer.

Motor Performance Evaluation. Motor coordination and balance were evaluated using the accelerating rotarod apparatus (Acuscan Instruments) (Jones, B. J. & Roberts, D. J. (1968) J. Pharm. Pharmacol. 20: 302-4; Sango, K., et al. (1996) Nature Genet. 14: 348-52). Mice were placed on a rotating dowel, and required to continuously walk forward to avoid falling off. The rod accelerated over 20 sec to a constant speed of 20 RPM and the latency to fall was recorded automatically by beams of light that, when broken, stopped a timer. Transgenic mice from the A2 and A6 lines were tested along with nontransgenic littermates. Mice were given 20 training trials, followed by 5 test trials, separated by 1 day between trials.

DNA Sequences. Plasmids containing the muscle creatine kinase promoter sequence and a cDNA encoding the human βAPP gene were provided by Drs. Stephen Hauschka and Rachel Neve, respectively. The sequence for the mouse muscle-specific form of the creatine kinase gene promoter is found at GenBank Accession No. AH001878. The sequence for human β-amyloid precursor protein is found at GenBank Accession No. XM047793.

### Results

Transgene Expression Analysis. To test the hypothesis that βAPP mismetabolism might play a causal role in the pathogenesis of IBM, transgenic mice were derived in which human βAPP expression was selectively targeted to skeletal muscle using the mouse MCK promoter (FIG. 1A). We selected this promoter because in other transgenic models it directed high expression levels to muscle, particularly to skeletal as compared to cardiac muscle (Johnson, J. E., Wold, B. J. & Hauschka, S. D. (1989) Mol. Cell. Biol. 9: 3393-9; Shield, M. A., et al. (1996) Mol. Cell. Biol. 16: 5058-68), and because expression is maintained throughout adulthood (Lyons, G., et al. (1991) Development. 113: 1017-29), an important criterion given that IBM is an age-related disorder. Five transgenic founder mice (referred to as A2, A4, A6, D10 and F9) identified by Southern blot analysis (data not shown) and were backcrossed to C57/BL6 mice. Three of the 5 lines (A2, A6 and D10) successfully transmitted the transgene to the F₁ generation.

To assay for transgene expression, we analyzed total RNA from skeletal muscle by Northern blot. To avoid visualizing endogenous mouse βApp mRNA, we used a probe targeted to the SV40 polyadenylation region because it was specific only for the transgene-produced transcript. We found that the A2 and A6 lines expressed the transgene in muscle to relatively low and high levels, respectively (FIG. 1B). Densitometric measurements of Northern blots indicated that the A6 line expressed the transgene approximately 12-times more than the A2 line (FIG. 1B). No evidence of transgene expression was found by Northern blot or by RT-PCR in the D10 line, so it was not characterized further.

Although other transgenic models achieved overexpression of the carboxy terminal 99-amino acids of βAPP in muscle, expression was under the control of a global promoter, and not restricted solely to muscle tissue (Fukuchi, K., et al. (1998) Am. J. Pathol. 153: 1687-93; Jin, L. W., et al. (1998) Am. J. Pathol. 153: 1679-86). To determine if βAPP transgene expression in the A2 and A6 lines was muscle specific, RNA from a variety of tissues including brain, intestine, kidney, liver, lung and stomach was analyzed by Northern blot. In both transgenic lines, MCK-directed transgene expression was exclusive to skeletal and cardiac muscle, with transgene mRNA levels approximately 8-times higher in skeletal muscle compared to heart (FIG. 1C). Notably, no transgene expression was detected in the non-target tissues (FIG. 1C). These findings are consistent with other transgenic studies that used this same promoter (Johnson, J. E., Wold, B. J. & Hauschka, S. D. (1989) Mol. Cell. Biol. 9: 3393-9; Shield, M. A., et al. (1996) Mol. Cell. Biol. 16: 5058-68).

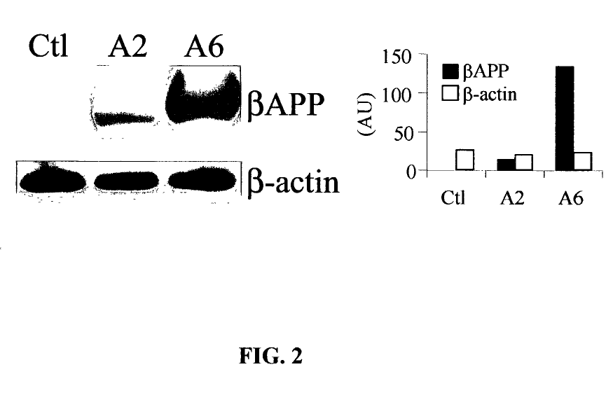

Steady-state levels of the transgene-derived human protein were determined by western blot analysis on total protein extracted from skeletal muscle of transgenic mice using an antibody that recognizes human but not murine βAPP. We detected human βAPP in protein extracts of skeletal muscle from both the A2 and A6 lines, with steady-state levels approximately 9-times higher in the A6 line compared to the A2 line (FIG. 2). As expected, we did not detect any human βAPP-immunoreactivity in skeletal muscle extracts from non-transgenic control littermates (FIG. 2), although we could detect endogenous βAPP using a murine specific antibody (data not shown).

IBM-like Histopathology in MCK-βAPP mice. Skeletal muscle samples from MCK-βAPP mice were histologically and immunohistochemically evaluated to determine the pathological consequences of human βAPP overexpression. In muscle from A2 or A6 mice that were less than 10 months of age, we did not detect any overt histopathological alterations. After this time period, however, we noted several IBM-like changes in the transgenic muscle, including centric nuclei, intracellular Aβ immunoreactivity, and cellular inflammation (Table 1). All of these histopathological changes were restricted to focal regions of the muscle tissue, in apposition to histologically-normal appearing fibers, consistent with the focal nature of the human disease.

TABLE 1
Summary of MCK-βAPP mice exhibiting IBM-like pathology
in skeletal muscle.

| | | Age in months | | | |
|---|---|---|---|---|---|
| Pathology | | 0-10 | 10-15 | 15-20 | 20-30 |
| Intracellular Aβ | A2 | 0/4 | 1/3 | 2/3 | 2/5 |
| immunoreactivity | A6 | 0/4 | 2/3 | 1/2 | 2/2 |
| Inflammation | A2 | 0/4 | 1/3 | 3/3 | 2/5 |
| | A6 | 0/4 | 1/3 | 1/2 | 2/2 |
| Centric | A2 | 0/4 | 0/3 | 2/3 | 2/5 |
| nuclei | A6 | 0/4 | 0/3 | 1/2 | 2/2 |

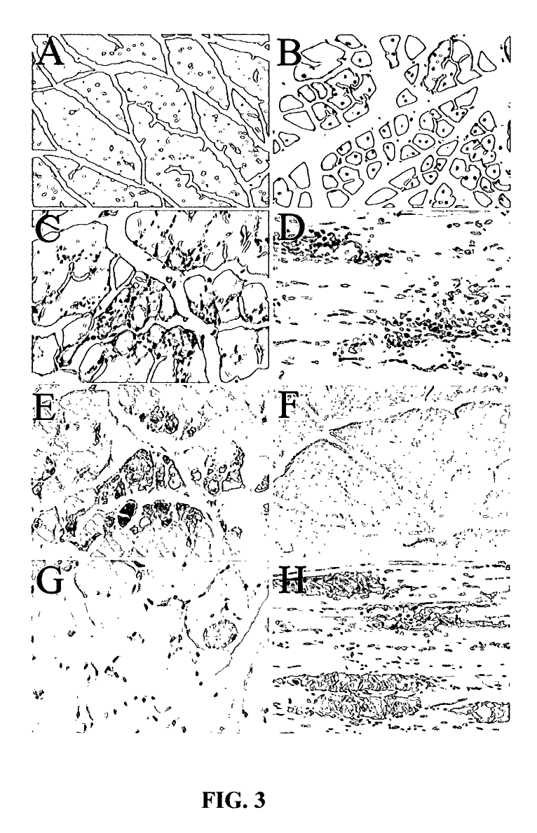

Representative HE-stained sections of skeletal muscle from a non-transgenic and transgenic mouse are shown (FIGS. 3A and B). Note that in the nontransgenic section, the nuclei are peripherally-located but in the transgenic tissue, they are located in the center of the muscle fiber. Centric nuclei are a general marker of muscle pathology often observed in muscle disorders, although they can occasionally be found in normal mouse and human tissue at a low frequency (1-3%). Adams, J. H. & Duchen, L. W. (1992) Greenfield's Neuropathology (Oxford University Press, New York). The occurrence of centric nuclei in a cluster of myofibers is indicative of underlying cellular damage and subsequent regenerative process in which the centric nuclei arise from newly recruited myoblasts to the site of degeneration. Hence, the occurrence of centric nuclei in focal regions of muscle in aged transgenic mice is consistent with the hypothesis that βAPP mismetabolism can lead to muscle fiber degeneration.

The presence of strongly basophilic mononuclear cells was another histopathological change observed in transgenic skeletal muscle. To identify the infiltrating cell type(s), tissue sections were immunostained with T-cell, macrophage-, and neutrophil-specific antibodies. We did not detect immunostaining with either the T-cell or macrophage-specific antibodies. In contrast, prominent neutrophil-specific immunoreactivity was evident in the transgenic tissue (FIG. 3D). The occurrence of inflammation represents an important component of our model, because it is a defining pathological characteristic of sporadic, but not hereditary, IBM (Askanas, V. & Engel, W. K. (1998) Curr. Opin. Rheumatol. 10: 530-42). In sporadic IBM, T-cell inflammation represents the major inflammatory cell type, but in our mouse model, neutrophils were the predominant cell type; the relevance of this disparity is presently unclear, but may be related to the expression of the transgene throughout ontogeny.

The presence of intramyofibril Aβ accumulation is a defining histopathological feature of IBM muscle biopsies and is used, in part, for its diagnosis (Askanas, V. & Engel, W. K. (1998) Curr. Opin. Rheumatol. 10: 530-42). In skeletal muscle tissue sections from aged (>12 months) mice of both the A2 and A6 lines, we detected Aβ immunoreactivity using several different Aβ antibodies (FIGS. 3E, G, H). As in human IBM, the Aβ immunoreactivity in the transgenic tissue was predominantly, if not exclusively, intracellular. Moreover, in the transgenic tissue, the Aβ immunostaining was not widely distributed throughout the tissue but was present in clusters of muscle fibers in focal regions. Notably, there was a strong overlap between regions showing immune infiltration and Aβ immunostaining as shown in serial sections (cf. 3C, E). Nevertheless, we could also detect intracellular Aβ immunoreactivity in regions without any prominent inflammation, consistent with the notion that accumulation of Aβ-containing fragments represents an early pathogenic event followed by the immune infiltration (data not shown). The intracellular Aβ immunoreactivity occurred in two distinct forms, as either amorphous diffuse structures (FIG. 3E, H) or as punctate granular deposits in ring-like patterns (FIG. 3G). Despite expression of the transgene in early-adulthood, we did not detect any Aβ immunoreactivity in muscle of transgenic mice less than 12 months of age. Likewise, we did not detect any Aβ immunostaining in nontransgenic muscle across all age groups examined (FIG. 3F).

ELISA Measurements. βAPP overexpression in muscle leads to development of IBM-like muscle pathology, which might be due to one of its various proteolytic derivatives. Levels of these βAPP proteolytic fragments were quantified using a well-characterized and highly sensitive ELISA system that can distinguish between steady-state levels of Aβ₄₀ and Aβ₄₂ (Murphy, M. P., et al. (2000) J. Biol. Chem. 275: 26277-84; Murphy, M. P., et al. (1999) J. Biol. Chem. 274: 11914-23; Suzuki, N., et al. (1994) Science 264: 1336-40; Kuo, Y. M., et al. (1996) J. Biol. Chem. 271: 4077-81; Scheuner, D., et al. (1996) Nat. Med. 2: 864-70; Lue, L. F., et al. (1999) Am. J. Pathol. 155: 853-62). Levels of Aβ₄₂ in the nontransgenic muscle samples were relatively low, ranging from 0.1-1.4 fmol/mg. Likewise, in the low expressing A2 transgenic line, Aβ₄₂ levels were marginally higher than age-matched nontransgenic controls, ranging from 0.3-3.1 fmol/mg. In the high expressing A6 line, however, Aβ₄₂ levels were markedly higher across all ages compared to both the A2 line and nontransgenic controls, ranging from 6.0-16.2 fmol/mg. There was no detectable Aβ₄₀ or Aβ₄₂ signal in formic acid fractions from either the A2 or A6 transgenic line (data not shown). There were no noticeable age-related differences for Aβ levels in either the controls or transgenics from the A2 or A6 line.

Because the Aβ antibodies used in the ELISAs can cross react with Aβ-containing fragments such as C99, we sought biochemical confirmation to determine the precise source of the Aβ signal observed. To assess this possibility, we immunodepleted the SDS-extracted skeletal muscle tissue with an antibody directed against the last 20 amino acids of βAPP. This antibody will efficiently immunoprecipitate either full-length βAPP or any βAPP fragments that contain these residues, including C83 and C99. SDS extracts “cleared” in this manner demonstrated markedly reduced (85%+/−7%; N=10) Aβ₄₂ readings. These findings indicate that there is a small amount of Aβ in these samples but that the predominant Aβ-containing fragment that appears to accumulate in the transgenic muscle is C99. Moreover, these findings along with the observation that no Aβ was detected in the formic acid fraction may explain why we failed to detect any congophilic-positive staining.

MCK-βAPP Mice Exhibit an Age-related Deficit in Rotarod Performance. Transgenic mice did not display any overt behavioral abnormalities nor was their life span diminished compared to nontransgenic control littermates. Because muscle weakness is the primary clinical component of IBM, we investigated whether deficits in muscle balance and coordination accompanied the underlying histopathological changes in MCK-βAPP muscle. Mice were evaluated on the accelerating rotarod, a well-characterized behavioral task designed to assess motor performance (Jones, B. J. & Roberts, D. J. (1968) J. Pharm. Pharmacol. 20: 302-4; Sango, K., et al. (1996) Nature Genet. 14: 348-52). At the earlier time points (<10 months of age) we did not detect significant differences in performance between transgenic and nontransgenic mice. In both the A2 and A6 lines, we noticed diminished performance between transgenic mice and age-matched controls beginning at 10-15 months of age. For the A6 line, these differences were statistically significant (p<0.05, Student's t-test) in mice beginning at 15-20 months of age when compared to nontransgenic control mice. Although transgenic mice from the A2 line displayed a similar trend towards an age-related decrease in their ability to remain on the rotarod compared to nontransgenic mice, these differences were not statistically significant. The relative differences in motor performance between A2 and A6 correlate with the low and high transgene expression levels observed between these two lines and the accumulation of increased βAPP proteolytic fragments.

### Discussion

Two independent transgenic mouse lines have been derived that selectively overexpress human βAPP in skeletal muscle. Notably, despite differing expression levels, both of the MCK-βAPP transgenic lines consistently developed a subset of IBM-like pathological changes in an age-dependent fashion that included (i) increased formation of Aβ-containing βAPP proteolytic fragments, (ii) a neutrophil-mediated inflammatory response that was clustered around βAPP immunoreactive regions, and (iii) deficits in motor performance. Although the etiology underlying sporadic IBM is not yet known, these data and those of others (Fukuchi, K., et al. (1998) Am. J. Pathol. 153: 1687-93; Jin, L. W., et al. (1998) Am. J. Pathol. 153: 1679-86) provide additional in vivo evidence implicating βAPP mismetabolism as an early, upstream event in the molecular pathogenesis of human IBM.

In the MCK-βAPP mice, IBM-like histopathological changes were observed in transgenic mice older than 10 months of age but not in younger transgenic or nontransgenic mice, mirroring the age-related aspect of the human condition. The motor deficits were also manifested as a function of age in our transgenic lines with the greatest decline occurring in the high expressing A6 line. Because the MCK promoter is transcriptionally active in young adult mice (Lyons, G., et al. (1991) Development. 113: 1017-29), it suggests that other unknown age-related factors are required for the elaboration of these phenotypic changes. It is clear, however, that these changes are not related to the site of integration because two independent transgenic lines were established that display a comparable phenotype. Likewise, despite expression of the transgene in every A2 or A6 mouse analyzed, not all aged transgenic mice developed intracellular Aβ immunoreactive deposits or other myopathological changes such as centric nuclei and atrophic fibers (see Table 1). Although we have no clear explanation for these observations, these results are consistent with previous studies showing that only a portion of transgenic mice that globally overexpressed the C100 fragment of βAPP, exhibited intramyofibril Aβ deposits (Fukuchi, K., et al. (1998) Am. J. Pathol. 153: 1687-93; Jin, L. W., et al. (1998) Am. J. Pathol. 153: 1679-86).

This transgenic model enables us to better understand the process by which accumulation of amyloidogenic fragments lead to cellular degeneration. In particular, because βAPP mismetabolism is involved in the pathogenesis of both Alzheimers Disease (AD) and IBM, insights learned about one disorder may also be applicable to both. An interesting distinction between the two disorders relates to the cellular location of the Aβ peptide, which is predominantly intracellular in IBM. Nevertheless, accumulating evidence suggests that intracellular Aβ may be an important and underappreciated component of Aβ pathology (LaFerla, F. M., et al. (1997) J. Clin. Invest. 100: 310-20; Gouras, G. K., et al. (2000) Am. J. Pathol. 156: 15-20; Tienari, P. J., et al. (1997) Proc. Natl. Acad. Sci. USA 94:4125-30; Skovronsky, D. M., Doms, R. W. & Lee, V. M. (1998) J. Cell. Biol. 141: 1031-9; Yang, A. J., et al. (1999) J. Biol. Chem. 274: 20650-6). Finally, given that no effective treatments exist for IBM patients, this mouse model offers the potential for evaluating the efficacy of novel therapeutics.

### Example 2

### Myofiber-Type Specific Accumulation of β-Amyloid Precursor Protein in a Transgenic Mouse Model of Inclusion Body Myositis

Skeletal muscle is a heterogeneous tissue containing subpopulations of muscle cells that differ in their ultrastructural, biochemical and physiological characteristics. The most significant differences that distinguish myofibers from one another are their rate of contraction and production and utilization of ATP as an energy source for contraction. Based upon these differences, skeletal muscle cells can be broadly categorized into two types: slow Type I and fast Type II (Schiaffino and Reggiani, 1994). The fast Type II fibers can be subdivided further, in rodents, into fast IIA, fast IIX and fast IIB (listed in order of ATPase activity) (Wakeland et al., 1990).

To investigate a potential molecular basis underlying the muscle pathological phenotype in the MCK-βAPP transgenic mice of Example 1, we determined the steady-state levels of human βAPP in different muscle groups and fiber types. As discussed herein, higher steady-state levels of the human βAPP were found within muscles comprised of a high percentage of fast-twitch fibers such as the white portion of the gastrocnemius (WMG) and plantaris (Pl). Single myofiber analyses revealed relatively higher steady-state levels of the human βAPP protein in fast Type IIB fibers compared to slow Type I fibers.

### Materials and Methods

Animals. Skeletal muscle protein for whole muscle and single myofiber detection of transgene protein were extracted from MCK-βAPP mice from the high-expressing A6 transgenic line of Example 1.

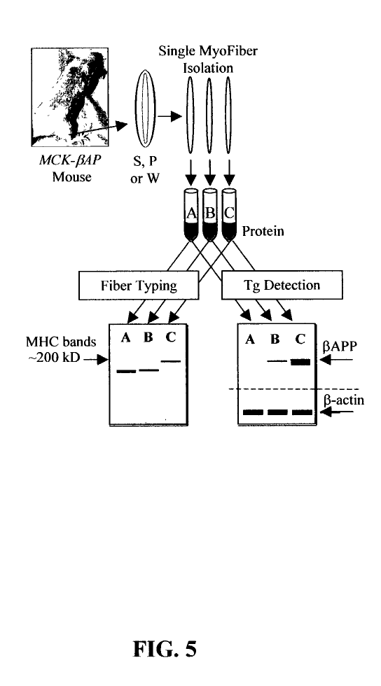

Isolation of single fibers. Following dissection, muscles were placed into glycerol relaxing solution (50% glycerol, 2 mM EGTA, 1 mM MgCl₂, 4 mM ATP, 10 mM imidazole, 100 mM KCl, pH 7.0) and stored overnight at −20° C. To obtain single fibers, muscles were placed in a small dissection chamber containing relaxing solution. Each fiber was transferred to individual tubes containing 30 μl of a running buffer: 62.5 mM Tris (pH 6.8), 1.0% (wt/vol) SDS, 0.01% (wt/vol) bromphenol blue, 15.0% (vol/vol) glycerol, and 5.0% (vol/vol) β-mercaptoethanol. To lyse the myofiber, each sample was heated (70° C. for 2 min) and vortexed for 15 sec. Approximately 15 μl of the protein extract was used for determining myosin heavy chain (“MHC”) isoforms by PAGE and the other 15 μl were used for detection of the transgene protein by Western immunoblotting. See FIG. 5 for a summary of the protocol used.

PAGE separation of MHC isoforms. MHC protein isoforms were separated using techniques described previously (Caiozzo et al., 1998; Caiozzo et al., 1997). The separating gel consisted of 8% acrylamide, 0.16% bis-acrylamide, 30% glycerol, 0.4% SDS, 0.2 M tris pH 8.8), and 0.1 M glycine. The solution was polymerized by adding N,N,N₈,N₈-tetramethylenediamine (0.05% final concentration) and ammonium persulfate (0.1% final concentration). The separating gel was poured, layered with ethyl alcohol, and given 30 min to polymerize.

The stacking gel solution contained 4% acrylamide, 0.08% bis-acrylamide, 30% glycerol, 70 mM Tris (pH 6.7), 4 mM EDTA, and 0.4% SDS. N,N,N₈,N₈-tetramethylenediamine (0.05% final concentration) and ammonium persulfate (0.1% final concentration) were added to polymerize the gel. It was then layered onto the separating gel. The running buffer contained 0.1 M Tris, 0.15 M glycine, and 0.1% SDS. Myofibril samples were denatured in sample buffer containing 5% β-mercaptoethanol, 100 mM Tris-base, 5% glycerol, 4% SDS, and bromophenol blue. Approximately 1 μg of protein was loaded into each well for protein extracted from whole muscles. An SG-200 vertical slab gel system (CBS Scientific, Del Mar, Calif.) was used for electrophoresis. Gels were run for ˜24 h at 270 V to separate fast type IIA, IIX, and IIB and the slow type I MHC isoforms (listed in order of migration). MHC protein isoform bands were visualized using a silver stain kit (Bio-Rad, Richmond, Calif.). A densitometer (Molecular Dynamics, Sunnyvale, Calif.) was used to scan and quantify the MHC isoform bands.

Western immunoblotting for transgene-derived human βAPP. The remaining half of single myofiber protein extract was utilized for transgene protein detection. Approximately 15 μl of the remaining protein extract was loaded into 7% Tris-Acetate mini gels (Invitrogen) and run at 150 V for 2 hours under reducing conditions. Protein was transferred for 1.5 hrs at 30 V onto nitrocellulose for immunoblotting. Membranes were immunoprobed with 6E10 antibody overnight at 4° C. with agitation as in Example 1. The bottom-half of the membrane was cut and immunoprobed for β-actin to confirm successful isolation of fibers and to normalize amount of protein levels from each fiber.

Histology. Skeletal muscle tissue dissected for histology was immediately frozen in liquid nitrogen cooled isopentane and stored at −80° C. Cryosections were cut at 10 Am, placed onto silane-coated slides and stored at −20° C. Hematoxylin-eosin (HE) staining was performed to determine general morphology of the muscle and single muscle fibers.

To localize the human βAPP protein, sections were immunostained using the P2-1 monoclonal antibody at a dilution of 1:250 (gift from Bill Van Nostrand). P2-1 recognizes an epitope at the N-terminus of the human βAPP protein, and will not cross-react endogenous mouse βAPP or with any human or mouse proteolytic fragments of βAPP such as C99 (Rozemuller et al., 1993). Sections were developed with diaminobenzidine substrate by using the avidin-biotin horseradish peroxidase system (Vector Laboratories) and counter-stained with hematoxylin.

To histologically examine the distribution of fiber types within each muscle, sections were stained for ATPase activity.

### Results

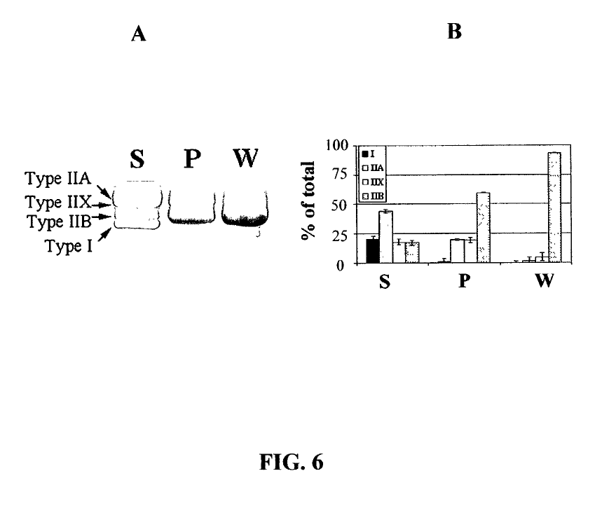

Distribution of MHC isoforms in mouse hind limb muscles. The Sol, Pl and WMG muscles of the distal hind limb were chosen based on their classifications of being slow, mostly fast and all fast muscle groups, respectively. Total protein was extracted from the Sol, Pl and WMG to determine relative levels of MHC isoforms in these hind limb muscles. To distinguish MHC isoforms, protein extracts were analyzed by SDS-PAGE. MHC isoforms migrate into 4 distinct bands corresponding to Type IIA, IIX, IIB and Type I, ranging from highest to lowest molecular weight, respectively (FIG. 6A).

Semi-quantitative analyses of MHC isoform bands by densitometric analyses revealed one half of MHC isoforms in the mouse Sol muscle was Type I with the other half being equally distributed between Types IIA, X and B isoforms. In the Pl, the majority of MHCs were comprised of Type IIB with scant evidence of the slow Type I isoform present. The WMG was comprised almost exclusively of Type IIB with little evidence of other MHC isoforms present (FIG. 6B). Although these data were obtained from transgenic mice, distribution of MHC isoforms in nontransgenic control mice was identical in Sol, Pl and WMG, indicating that βAPP did not disrupt the overall distribution of the MHC isoforms within these muscles.

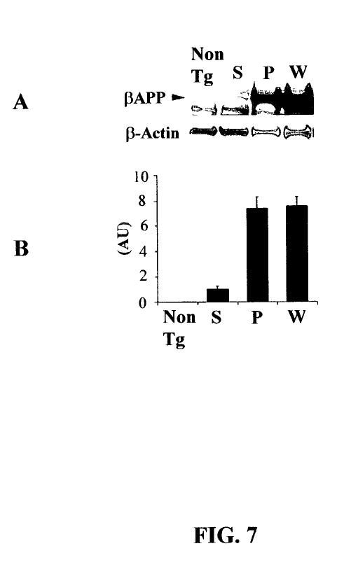

βAPP is differentially expressed in skeletal muscle of MCK-βAPP transgenic mice. Total protein extracted from the Sol, Pl, and WMG from five A6 transgenic mice were immunoblotted to determine the steady-state levels of the transgene protein. High steady-state levels of human protein were detected in the Pl and the WMG, with relatively lower levels found in the Sol (FIG. 7A).

The blot was re-immunoprobed for β-actin to normalize for amount of protein loaded (FIG. 7A). Higher levels of human βAPP were found in muscles with a higher proportion of fast MHC isoforms, which suggests several possibilities: (i) higher steady-state levels of human βAPP in fast versus slow fibers; (ii) a preferential accumulation of the protein, or; (iii) a failure to clear accumulated βAPP.

Single-fiber analyses for transgene protein and MHC isoform. Prior results indicated a disparity in the steady-state levels of βAPP in different skeletal muscle groups (FIGS. 7A,B) Relatively higher levels are detected in muscle groups that contain a high proportion of fast MHC isoforms (FIGS. 6 and 7).

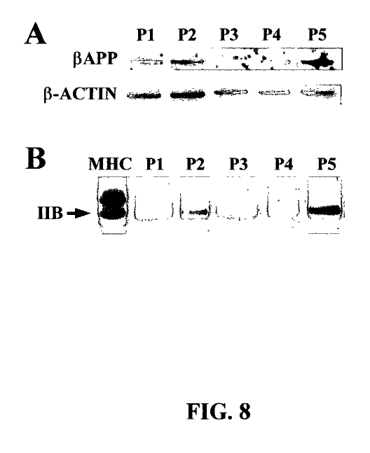

To better characterize the molecular profile in this mouse model, human βAPP protein levels were analyzed in single muscle fibers and correlated with their predominant respective MHC isoform. (FIG. 8) These analyses allow for a more precise quantitation of levels of βAPP between fast and slow fiber types.

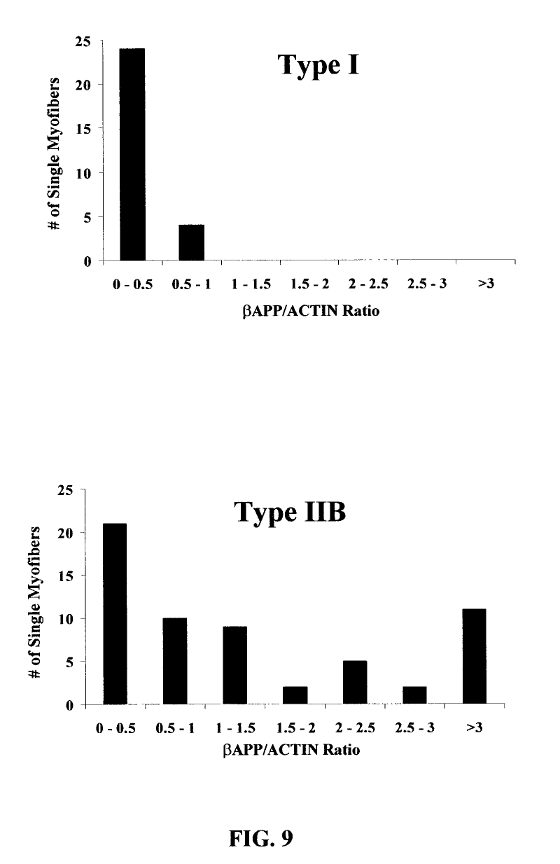

Nearly two-thirds of the total number of Type I fibers analyzed either contained barely detectable levels of transgene protein or nothing at all. As shown in FIG. 9, human βAPP levels in Type IIB fibers were higher compared to the Type I fibers.

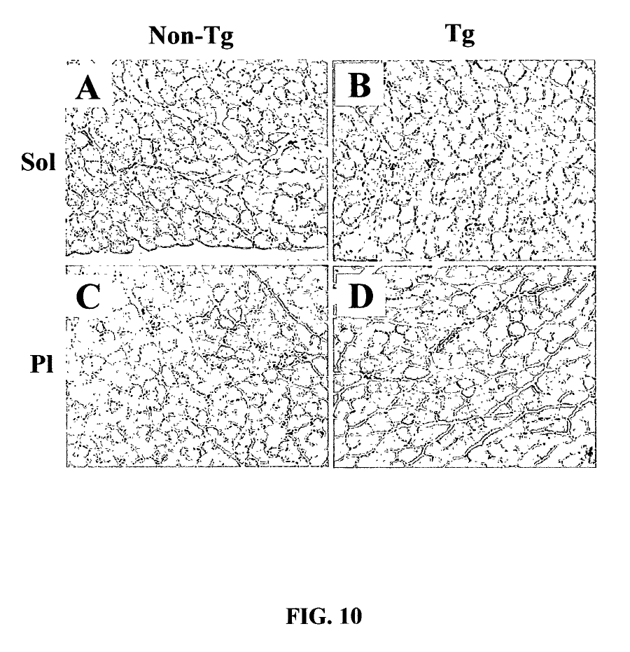

Immunohistochemical detection of human βAPP in muscle of MCK-βAPP mice. To histologically localize human βAPP protein, cryosections from the Sol and Pl from transgenic mice were immunostained using the P2-1 monoclonal antibody, which is a human-specific monoclonal antibody (Rozemuller et al., 1993). Thus, no immunoreactivity was detected in nontransgenic control Sol and Pl skeletal muscle sections (FIGS. 10, A and C). Prominent immunostaining was observed in 9-month old A6 transgenic Pl skeletal muscle tissue (FIG. 10D). Although protein extracts from the whole Sol muscle of a transgenic mouse revealed low levels of transgene protein by Western blotting (FIG. 7), it was not detected via immunohistochemistry (FIG. 10B).

### Discussion

Pathological processes that underlie various skeletal muscle diseases may differentially affect fast and slow twitch muscle fibers. For example, Type II specific atrophy is observed in the skeletal muscle of patients with myasthenia gravis, as well in conditions of protein malnutrition and chronic high-dose use of corticosteroids (Carpenter and Karpati, 2001). Moreover, Type I specific atrophy occurs in several childhood neuromuscular diseases, including infantile myotonic dystrophy (Carpenter and Karpati, 2001). Determining whether a particular muscle type is preferentially affected is key to elucidating the pathogenic mechanisms that underlie the disorder. In addition, identifying the muscle type that is selectively affected can be useful for charting out the disease progression and facilitating the development of appropriate therapeutics. In human IBM, it remains to be established if fast and slow twitch fibers are differentially affected.

High steady-state levels of transgene βAPP were present in skeletal muscles that contain relatively high levels of fast Type II MHC isoforms, such as the WMG and Pl muscles (FIGS. 6, 7), whereas low levels of the transgene protein were found in slow muscles such as the Sol (FIGS. 6, 7). These data clearly show that transgene protein is not equally distributed in all muscle groups examined.

To quantitatively investigate the distribution profile of the transgene protein among different muscle fiber types, single muscle fibers were microdissected from transgene muscle and analyzed for the presence of the transgene protein and the predominant MHC isoform. These studies revealed high levels of transgene protein in Type IIB fibers compared to Type I slow fibers (FIGS. 8-10).

As a final note, although the role of endogenous βAPP in skeletal muscle is still unknown, βAPP localizes to the neuromuscular junction (NMJ) in the developing and mature rodent, suggesting a possible role in nerve-muscle communication (Akaaboune et al., 2000). Thus, overexpression and accumulation of the βAPP protein may result in aberrant NMJ function, which may be investigated using the MCK-βAPP mice as well as in human IBM.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

What is claimed is:

1. A transgenic mouse whose genome comprises a transgene encoding human β-amyloid precursor protein (“βAPP”), wherein the transgene is operably linked to a muscle-specific promoter, and wherein expression of the transgene results in increased formation of Aβ-containing βAPP proteolytic fragments.

2. The transgenic mouse of claim 1, wherein the βAPP transgene comprises a complete cDNA βAPP sequence.

3. The transgenic mouse of claim 1, wherein the expression of the transgene is limited to skeletal muscle.

4. The transgenic mouse of claim 1, wherein the mouse is fertile and transmits the βAPP transgene to its offspring.

5. The transgenic mouse of claim 1, wherein the βAPP transgene has been introduced into an ancestor of said mouse at an embryonic stage.

6. The transgenic mouse of claim 1, wherein the mouse is heterozygous for the human βAPP transgene.

7. The transgenic mouse of claim 1, wherein the mouse is homozygous for the human βAPP transgene.

8. The transgenic mouse of claim 1, wherein the mouse overexpresses βAPP in muscle tissue relative to a control non-transgenic mouse.

9. The transgenic mouse of claim 1, wherein the human βAPP transgene is a mutant βAPP.

10. The transgenic mouse of claim 1, wherein the promoter is a muscle creatine kinase promoter.

11. A transgenic mouse whose genome comprises a transgene encoding full-length β-amyloid precursor protein, wherein the transgene is operably linked to a muscle-specific promoter, and wherein expression of the transgene results in a neutrophil-mediated inflammatory response clustered around βAPP immunoreactive regions.

12. The transgenic mouse of claim 11, wherein the βAPP transgene comprises a complete cDNA βAPP sequence.

13. The transgenic mouse of claim 11, wherein the expression of the transgene is limited to skeletal muscle.

14. The transgenic mouse of claim 11, wherein the mouse is fertile and transmits the βAPP transgene to its offspring.

15. The transgenic mouse of claim 11, wherein the βAPP transgene has been introduced into an ancestor of said mouse at an embryonic stage.

16. The transgenic mouse of claim 11, wherein the mouse is heterozygous for the human βAPP transgene.

17. The transgenic mouse of claim 11, wherein the mouse is homozygous for the human βAPP transgene.

18. The transgenic mouse of claim 11, wherein the mouse overexpresses βAPP in muscle tissue relative to a control non-transgenic mouse.

19. The transgenic mouse of claim 11, wherein the human βAPP transgene is a mutant βAPP.

20. The transgenic mouse of claim 11, wherein the promoter is a muscle creatine kinase promoter.

21. A transgenic mouse whose genome comprises a transgene encoding full-length β-amyloid precursor protein, wherein the transgene is operably linked to a muscle-specific promoter, and wherein expression of the transgene results in deficits in motor performance.

22. The transgenic mouse of claim 21, wherein the βAPP transgene comprises a complete cDNA βAPP sequence.

23. The transgenic mouse of claim 21, wherein the expression of the transgene is limited to skeletal muscle.

24. The transgenic mouse of claim 21, wherein the mouse is fertile and transmits the βAPP transgene to its offspring.

25. The transgenic mouse of claim 21, wherein the βAPP transgene has been introduced into an ancestor of said mouse at an embryonic stage.

26. The transgenic mouse of claim 21, wherein the mouse is heterozygous for the human βAPP transgene.

27. The transgenic mouse of claim 21, wherein the mouse is homozygous for the human βAPP transgene.

28. The transgenic mouse of claim 21, wherein the mouse overexpresses βAPP in muscle tissue relative to a control non-transgenic mouse.

29. The transgenic mouse of claim 21, wherein the human βAPP transgene is a mutant βAPP.

30. The transgenic mouse of claim 21, wherein the promoter is a muscle creatine kinase promoter.

31. A method of screening biologically active agents that facilitate reduction of Aβ-containing βAPP proteolytic fragments in vivo, the method comprising:
administering a candidate agent to a transgenic mouse according to claim 1, and
determining the effect of said agent upon the amount of Aβ-containing βAPP proteolytic fragments.

32. A method of screening biologically active agents that facilitate reduction of a neutrophil-mediated inflammatory response clustered around βAPP immunoreactive regions in vivo, the method comprising:
administering a candidate agent to a transgenic mouse according to claim 11, and
determining the effect of said agent upon the amount of the neutrophil-mediated inflammatory response.

33. A method of screening biologically active agents that facilitate improvement in motor performance, the method comprising:
administering a candidate agent to a transgenic mouse according to claim 21, and
determining the effect of said agent upon motor performance.
